(19) United States
(12) Patent Application Publication
(10) Pub. No.: US 2009/0012204 A1
(43) Pub. Date: Jan. 8, 2009

(54) FUNCTIONALIZATION OF POLYMERS WITH REACTIVE SPECIES HAVING BOND-STABILIZED DECONTAMINATION ACTIVITY

(75) Inventors: Ulf Drechsler, Bryan, TX (US); Waheguru Singh, College Station, TX (US); Anjal Sharma, College Station, TX (US)

Correspondence Address: STREETS & STEELE, 13831 NORTHWEST FREEWAY, SUITE 355, HOUSTON, TX, 77040, US

(73) Assignee: LYNNTECH, INC., College Station, TX (US)

(21) Appl. No.: 12/166,254

(22) Filed: Jul. 1, 2008

Related U.S. Application Data
(60) Provisional application No. 60/948,275, filed on Jul. 6, 2007.

(51) Int. Cl. A61K 8/73; C08B 31/00; C08G 18/00; C08G 77/14

(52) U.S. Cl. 523/105; 525/54.3; 525/54.31; 528/44; 528/28

(57) ABSTRACT

Functionalized polymers and methods of functionalizing polymers with reactive species having decontaminating activity, such as polyoxometalates and metal oxides. Covalent bonding of the reactive species to the polymer securely immobilizes the reactive species and stabilizes the decontaminating activity of the reactive species. Specifically, the covalent bonding of the reactive species greatly reduces moisture deactivation during prolonged exposure to atmospheric moisture. Polyoxometalates are catalytically reactive through oxidative pathways and metal oxides are reactive through hydrolytic pathways. Both polyoxometalates and metal oxides having oxygen atoms available for covalent bonding with an appropriate bifunctional linking agent.

### CROSS-REFERENCE TO RELATED APPLICATIONS

This application claims priority of U.S. Provisional Patent Application Ser. No. 60/948,275 filed on Jul. 6, 2007.

### STATEMENT REGARDING FEDERALLY SPONSORED RESEARCH OR DEVELOPMENT

This invention was made with government support under grant number 1 R43 AI058634-01 awarded by the National Institutes of Health (NIH) and contract number W911QY-07-C-0004 awarded by the Department of Defense (Army). The government has certain rights in this invention.

### BACKGROUND OF THE INVENTION

1. Field of the Invention

The present invention relates to the functionalization of a polymer with reactive species to achieve decontaminating activity.

2. Background of the Related Art

Most chemical protective clothing is really “chemical-resistant” in that they merely provide a physical barrier to inhibit penetration of toxic agents through the fabric thereby protecting the wearer. Such garments are almost invariably of thick construction and heavy in weight, and are often fabricated at least in part from materials impermeable to water or water vapor, such as natural and synthetic rubbers and elastomers, chlorinated rubbers, etc. These garments place heavy burdens on those who wear the garments by restricting heat dissipation through the natural evaporation of sweat, as well as restricting movement due to the bulkiness and weight of the material. In order to solve these problems, suits are now being made that allow free airflow through pores while selectively removing or trapping certain toxic components.

While air-permeable chemical-protective clothing reduces the problem of heat dissipation by sweat evaporation, it has the inherent drawback of being permeable to hazardous vapors, aerosols, and particulate materials. Furthermore, clothes that are “breathing” are characterized by intrinsic bulkiness due to the fact that they are designed for carrying relatively large loads of adsorbent material required to provide protection against toxic chemicals during a reasonably sufficient period of time. It is also recognized that the breathing materials also do not adequately solve the physiological load and heat stress problems of the chemical protective garments, and they may also lead to incapacitation, thermal shock, and even death under conditions of severe workloads, and high temperatures and humidity. Protective clothing made of laminates of films has the problem of forming “kinks” when bent so as to restrict movement and become cumbersome.

Moreover, some protective clothing is porous and provides little protection against hazardous chemical vapors. The major disadvantage with these existing materials is that they either provide a barrier to the toxic chemicals or they absorb them, but they do not have the ability to decontaminate the toxic agents. Therefore, the clothing itself becomes a hazardous waste material. Even if the clothes can be decontaminated using various chemical agents, the runoff from decontamination must be disposed of as well. Furthermore, the material comprising most of these garments are not biodegradable, which raises environmental and waste disposal concerns. Therefore, barrier articles or clothing that absorbs chemical agents are not an answer to the problem.

Clothing has also been made by trapping known antimicrobial agents in a fibrous matrix. However, most of the antimicrobial compounds used in these finishing processes, whether physically dispersed or covalently attached to fabrics, have specific mechanisms of antimicrobial action that can spur the development of resistant microorganisms. In addition, none of the fabric materials containing these agents can be regenerated. Therefore, after the antimicrobial properties of these fabrics have been exhausted, they can no longer be used.

Other researchers have found ways to covalently link antimicrobial agents to textiles so that the agent will be permanently bonded to the fabric thereby preventing leaching of the antimicrobial compound into the surrounding environment. In U.S. Pat. No. 5,855,987, Margel et al. synthetically immobilized antimicrobial enzymes such as lysozyme and chymotrypsin to cellulose thereby rendering the fabric antimicrobial.

In U.S. Pat. No. 5,882,357, Sun et al. describes a finishing process for textiles where a heterocyclic N-halamine is covalently attached to cellulose-based fabrics to impart biocidal activity. This biocidal activity can be regenerated after exhaustion by reacting the fabric with a halogenated solution.

However, there remains a need for a material exhibiting self-decontaminating capabilities to deactivate a variety of contaminants, such as chemical warfare agents and other toxic chemicals, and also having antimicrobial properties. It would be desirable if the material had the ability to destroy pathogenic microorganisms, harmful chemical compounds and biological/chemical warfare agents. Ideally, these properties would be provided to a fabric while retaining the desired physical properties of the starting fabric. It would be even more desirable to have a method of treating fabric to produce protective clothing having a long lifetime.

### SUMMARY OF THE INVENTION

The present invention provides a polymer material and a method of modifying a polymer. The polymeric material comprises at least one reactive species covalently linked to a polymer, wherein the reactive species is selected from the group consisting of heteropolyacid, polyoxometalate, metal oxide, and combinations thereof. When the reactive species includes a metal oxide, such as TiO₂, MgO, ZnO, CaO, Al₂O₃, and combinations thereof, the metal oxide may be linked through a trialkoxysilyl group, such as (triethoxysilyl)propyl isocyanate. Preferably, the metal oxide will be provided as nanoparticles having a particle size between 1 and 100 nanometers. When the reactive species includes heteropolyacid or polyoxometalate, such as H₅PV₂Mo₁₀O₄₀, Ag₅PV₂Mo₁₀O₄₀, and H₃PMo₁₂O₄₀ and combinations thereof, the heteropolyacid or polyoxometalate may be linked with a diisocyanate, such as toluene diisocyanate. In one embodiment, the at least one reactive species includes both a polyoxometalate and a metal oxide, and wherein the at least one linking agent includes both a diisocyanate and an isocyanate/alkoxysilane. Examples of polymers to which the reactive species is covalently linked include polyethylene, polypropylene, polyester, polyamide, polyacrylonitrile, polyurethane, polyvinyl alcohol, polyethylene imine, polypropylene imine, and polysaccharides. It has been found that the stability of the reactive species against moisture based deactivation is increased by the covalent bond formed with the polymeric material and that the polymeric material is non-cytotoxic. Optionally, silver ions may be immobilized on the reactive species and/or a dye may be incorporated onto the polymer, wherein the dye exhibits a change in color in response to a change in pH caused by reacting a contaminant species at the reactive species.

The method of modifying a polymer, comprises reacting a first reactive functional group of at least one bifunctional linking agent with a reactive moiety of the polymer to form a covalent bond therebetween, and reacting at least one oxygen atom of at least one reactive species with a second reactive functional group of the at least one bifunctional linking agent to form a covalent bond therebetween. The reactive species may include heteropolyacid, polyoxometalate, metal oxide or a combination thereof. The method may be performed on a polymer that has already been formed into a textile.

When the reactive species includes heteropolyacid, polyoxometalate, or a combination thereof, then the first and second reactive functional groups may be independently selected from the group consisting of isocyanate, isothiocyanate, acyl halide, carboxylic anhydride, carboxylic acid, ester, alkyl halide, oxirane, and oxetane. A suitable polyoxometalate may be selected from the group consisting of Keggin-type polyoxometalates defined by the general formula (R^{m+})_{y}[X^{a+}M₁₂O₄₀]⁽⁸⁻ⁿ⁾⁻ where m and n are an integer, y is defined as y=(8−n)/m, X is selected from phosphorus and silicon, M is selected from molybdenum, tungsten, vanadium, and combinations thereof, O is oxygen, and R is selected from the group consisting of hydrogen, silver, ammonium, quaternary ammonium, and combinations thereof. Specific examples of the polyoxometalate include H₅PV₂Mo₁₀O₄₀, Ag₅PV₂Mo₁₀O₄₀, and H₃PMo₁₂O₄₀ and combinations thereof. The bifunctional linking agent may be a diisocyanate, such as an aromatic diisocyanate selected from the group consisting of 2,4-toluene diisocyanate, 4,4′-methylenebis(phenyl isocyanate) and combinations thereof, or an aliphatic diisocyanate selected from the group consisting of 4,4′-methylenebis(cyclohexyl isocyanate), tetramethylxylene diisocyanate and combinations thereof.

When the reactive species includes a metal oxide, then the second reactive functional group is preferably a trialkoxysilyl group, such as (triethoxysilyl)propyl isocyanate. The first reactive functional group may be selected from the group consisting of isocyanate, isothiocyanate, acyl halide, carboxylic anhydride, carboxylic acid, alkyl halide, oxirane, oxetane and combinations thereof. Examples of suitable metal oxides are selected from the group consisting of TiO₂, MgO, ZnO, CaO, Al₂O₃, and combinations thereof. It is preferable that a majority of the metal oxide nanoparticles have a particle size between 1 and 100 nanometers.

Examples of suitable polymers include polyvinyl alcohol, polyethylene imine, polypropylene imine, silica and functionalized silica based polymers, and polysaccharides, such as cellulose. In one embodiment, the method further comprises hydroxylating the polymer through exposure to an oxygen plasma or ozone. This is necessary when the polymer is selected from the group consisting of polyethylene, polypropylene, polyester, polyamide, polyacrylonitrile, polyurethane, and combinations thereof.

In a further embodiment, the at least one reactive species includes both a polyoxometalate and a metal oxide, and wherein the at least one linking agent includes both a diisocyanate and an isocyanate/alkoxysilane. Preferably, the diisocyanate and the isocyanate/alkoxysilane are simultaneously reacted with the reactive moiety of the polymer to form an activated polymer. It is also preferable to react the metal oxide with alkosysilane groups of the activated polymer before reacting the polyoxometalate with isocyanate groups of the activated polymer.

Optionally, silver ions may be immobilized on the reactive species, such as by electrostatically immobilizing the silver ions by contacting the reactive species-linked polymer in an aqueous silver nitrate solution. In a further optional embodiment, an indicator dye may be incorporated onto the polymer, wherein the dye exhibits a change in color in response to a change in pH caused by reacting a contaminant species at the reactive species. Covalently linked polyoxometalates or metal oxide nanoparticles can directly provide a color change to the fabric upon exposure to chemical agents due to the decontamination reaction, thus helping with agent identification.

### BRIEF DESCRIPTION OF THE DRAWINGS

FIG. 1 is a process diagram describing the steps for processing a polymer.

FIG. 2 is a process flow diagram of the steps involved in grafting polyoxometalate onto a hydroxyl-containing polymer and depositing silver on the polyoxometalate.

FIG. 3 is a graph showing the cumulative breakthrough behavior for CEES and DEMP on POM modified cotton (no silver) compared to unmodified cotton as control.

FIG. 4 is a graph of the cumulative breakthrough behavior for CEES and DEMP on POM modified fabric made from a rayon/polypropylene blend.

FIG. 5 is a graph showing that breakthrough protection is completely preserved after storage for several weeks.

FIG. 6 is a bar graph of the kill rate of P. aeruginosa and S. aureus as a function of silver nitrate concentration after 20 minutes.

FIG. 7 is a graph showing the breakthrough behavior of diethyl methyl phosphonate (DEMP), a G agent surrogate, using metal oxide nanoparticle attached cotton swatches.

FIG. 8 is a graph of the cumulative breakthrough behavior for 2-chloroethyl ethyl sulfide (CEES), an H agent surrogate, on metal oxide nanoparticle modified cotton cloth compared to unmodified cotton as a control.

FIG. 9 is a graph of the cumulative breakthrough behavior for a metal oxide modified polypropylene material.

FIG. 10 is a graph of the CEES decontamination behavior for a NP and POM modified polyurethane wipe over a 30 minute period, as compared with a control.

FIG. 11 is a graph of the DEMP decontamination behavior for a NP and POM modified polyurethane wipe over a 30 minute period, as compared with a control.

FIG. 12 is a schematic diagram of the reaction of hydroxyl groups naturally present on cellulose fibers with a 2,4-toluene diisocyanate cross linker, subsequent attachment of polyoxometalate on the activated fiber, and the ion exchange of THMP onto the POM.

### DETAILED DESCRIPTION OF THE INVENTION

The present invention provides a method of functionalizing polymers with a reactive species having decontaminating activity. According to the method, covalent bonding of the reactive species to the polymer securely immobilizes the reactive species and stabilizes the decontaminating activity of the reactive species. Specifically, the covalent bonding of the reactive species in accordance with the invention has shown the ability to prevent moisture deactivation during prolonged exposure to atmospheric moisture.

The reactive species is a polyoxometalate in protonated or unprotonated form, a metal oxide, or a combination thereof. Polyoxometalates in protonated or unprotonated form are catalytically reactive through oxidative pathways and metal oxides are reactive through hydrolytic pathways, but they are both useful for destroying various contaminants. In fact, using a combination of polyoxometalates and metal oxides can decontaminate a very broad range of chemical and/or biological contaminants due to the combination of oxidative and hydrolytic reaction pathways. Furthermore, both polyoxometalates and metal oxides have oxygen atoms available for covalent bonding with an appropriate linking agent.

Polyoxometalates are a class of inorganic cluster compounds comprised of numerous oxygen atoms, sometimes hydrogen atoms, and atoms of at least two other elements in positive oxidation states. Keggin-type heteropolyanions (the unprotonated form of polyoxometalates) are the most widely studied polyoxometalates and are defined by the general formula [Xⁿ⁺M₁₂O₄₀]⁽⁸⁻ⁿ⁾⁻ where the heteroatom X can be one of many d and p elements, mostly phosphorus or silicon, and M can be one or more transition metal including molybdenum, tungsten, vanadium, and others. A few non-limiting examples of polyoxometalates include (Bu₄N)₅PV₂Mo₁₀O₄₀ and Ag₅PV₂Mo₁₀O₄₀, H₅PV₂Mo₁₀O₄₀, and H₃PMo₁₂O₄₀. Many examples of polyoxometalates undergo rapid, reversible redox changes. These compounds have been widely used as both oxidation and acid catalysts due to their high oxidation potential and strong acidity. Polyoxometalates are inexpensive and easy to synthesize, often through a one-pot synthesis. Virtually all molecular properties including composition, size, shape, charge density, redox potentials, acidity, and solubility can be varied by simply modifying the well-established synthetic routes to meet the requirements of a specific application.

Many polyoxometalates are excellent electron transfer agents in the selective catalytic oxidation of inorganic and organic substrates, capable of oxidizing many organic compounds including chloroaromatics, herbicides, alcohols, organic acids, ketones, and esters either in solution or supported on several matrices such as, for example, silica and zeolites. Polyoxometalate-mediated oxidation reactions can be catalytic, polyoxometalates are reduced as a result of this oxidation reaction, and the reduced polyoxometalates can be re-oxidized to their original state by oxygen or other suitable oxidants in the system, completing the catalytic cycle.

Metal oxides, such as TiO₂, MgO, ZnO, CaO and Al₂O₃, are also suitable reactive species that can be covalently linked to a polymer in accordance with the invention. The covalent bonding has been found to greatly improve the stability of metal oxides against deactivation by atmospheric moisture. Accordingly, the metal oxides retain their activity over time and are capable of causing hydrolytic decomposition of contaminants.

Metals and their oxides with individual particle sizes 1 to 100 nm range are quite different from bulk material in terms of their optical, electrical, magnetic, and physical properties. Nanometer-sized metal oxide particles possess high surface areas (up to 600 m²/g has been reported), many pores, and unsaturated surface cations and anions with chemical reaction capabilities. Several approaches to the synthesis of nanometer-sized inorganic materials are known, including metal ion reduction in the presence of surfactants, organic capping agents, polymers, and dendrimers. Another approach to producing these particles is to precipitate metal ions in aqueous media to form a hydroxide gel and utilizing an aerogel process that produces particularly useful materials. Metal oxide aerogels are low bulk density materials with very large surface areas and large pore volumes and consists of very small weakly aggregated crystallites (about 4 to 7 nm in size), as described in U.S. Pat. No. 5,759,939, which patent is incorporated by reference herein. These nanometer-sized metal oxide particles are commercially available from NanoScale Materials, Inc. (Manhattan, Kans.). Metal oxide nanoparticles such as TiO₂, MgO, ZnO, CaO and Al₂O₃, have found uses as adsorbents, catalysts, biocides, and especially as decontaminants of CWAs and surrogates.

In one embodiment, dyes that undergo a color change in the presence of contaminants or their breakdown products may be co-immobilized with the reactive species to incorporate contamination-indicating properties to the composition. Metal oxide nanoparticles such as TiO₂, MgO, ZnO, CaO and Al₂O₃ hydrolyze many CWAs into acidic breakdown products. The acid thus generated induces a color change of a co-immobilized pH-sensitive dye. A few non-limiting examples of preferred pH-sensitive dyes include methyl red, methyl orange, and bromophenol blue. The well-known color change of certain polyoxometalates such as phosphomolybdic acid undergoing changes in oxidation states as a result of a redox reaction may also be utilized as indication of the presence of contaminants. Incorporation of a dye may include either physical adsorption or covalent attachment. The change in color of dye can be also made independent of decontamination reaction and pH change if we use Solvatochromic dyes for toxic chemicals identifications. In a related embodiment, a color change can take place due to a decontamination reaction even without the use of a dye in case of a POM modified fabric.

In one embodiment, silver may be deposited on the reactive species to incorporate antimicrobial activity to the composition. The diversity of application of silver based biocides is not only due to their low toxicity to human cells, but also to their effectiveness against broad range of microbes. The antimicrobial spectrum of silver is impressive including Gram-negative bacterial species such as Escherichia coli, Enterobacter, Klebsiella, and Pseudomonas, as well as Gram-positive bacterial species such as Candida albicans. Silver has also been reported to be virucidal against poliovirus, adenovirus, bovine rotavirus, herpes, and vaccina virus. Surprisingly, the inclusion of silver does not appear to deter the performance of either an immobilized polyoxometalate or an immobilized metal oxide.

Immobilization of silver with polyoxometalates will also lead to high local density of silver ions, resulting in better biocidal properties since one individual polyoxometalate cluster can form a stable complex with up to five silver ions.

The polymers to which the reactive species are linked are preferably selected or modified to include chemically reactive moieties, such as free hydroxyl, amino, aldehyde, ketone, carboxylic acid, and other groups. The preferred hydroxyl groups provide an active hydrogen atom that facilitates covalent bonding with linking agents. Cellulose-based fabric, such as cotton and rayon, as well as silica and functionalized silica polymers inherently include hydroxyl groups. Many other polymers, such as polyethylene, polypropylene, polyester, polyamide, polyacrylontrile, or polyurethane, do not inherently contain reactive groups such as hydroxyls, and must be modified prior to reaction with a linking agent. For example, ozone or oxygen plasma treatment is used to produce surface hydroxyl groups on these polymers. Advantageously, the process of modifying a polymer to include hydroxyl groups and/or the process of linking reactive species onto the surface of a hydroxyl-containing polymer can be performed on a prefabricated article, such as a textile or foam.

In one embodiment, the polymer is in the form of a polyurethane sponge material that is processed according to the invention to include covalently attached and stabilized polyoxometalates and metal oxides. These polymeric materials can be tailored in to a variety of cleaning products (wipes, mittens, towels, drapes, covers, etc.) suitable for cleaning up chemicals or biological matter. These materials can also be used to create air filters, protective clothing and respiratory masks for protection from chemical agents and toxic industrial chemicals.

In yet another embodiment, a textile material, such as cotton, rayon or a cotton/polyester blend, is modified with polyoxometalates to serve as a permanently attached flame retardant. The resulting textile material can withstand harsh conditions without leaching of the retardant. Any of the methods described herein may be used to permanently attach the polyoxometalates. Polyoxometalate-modified textiles provide several valuable flame retardant properties, including reduced flammability compared to the unmodified textile material, reduced (or at least not increased) smoke generation, no increased toxicity of combustion products from the functionalized textile material compared to the unmodified one, durability retained in the product through normal use (including outdoor exposure, laundering), acceptable minimal effect on other physical properties (mechanical strength, flexibility, breathability etc.) of the unmodified textile material, no skin toxicity, and acceptable cost. Optionally, once the POM is chemically fixed to the textile, additional flame retardant chemicals can be attached. For example, positively charged phosphonium ions (e.g., tetrakis(hydroxymethyl)-phosphonium, THMP) will form an ion pair with the negatively charged oxygen groups in the POM.

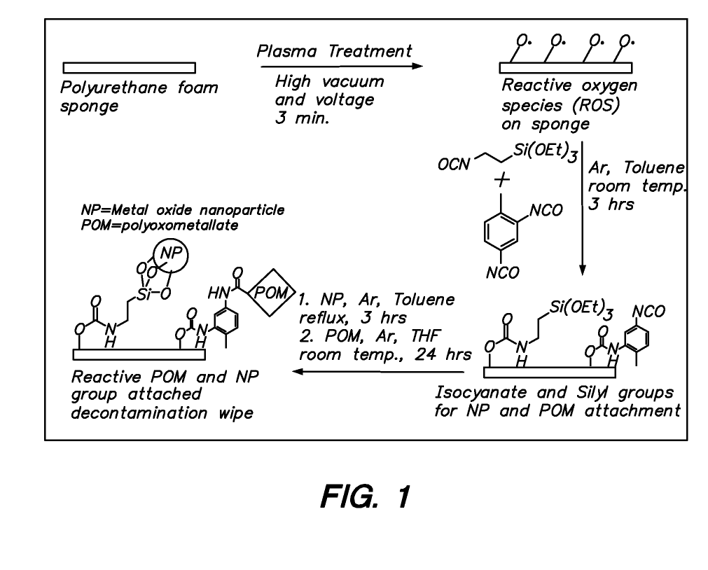

FIG. 1 is a process diagram describing the steps for processing a polymer. The process as shown includes various aspects of the invention that are not necessary to every embodiment of the invention. For example, the diagram includes a plasma treatment that is only beneficial if the polymer substrate does not inherently include a reactive functional group. Furthermore, the diagram includes the covalent bonding of both a metal oxide and a polyoxometalate, whereas the invention includes embodiments that only bond a single reactive species.

Linking agents are selected having a first functional group that reacts with the reactive functional group and form a covalent link to the polymer and a second functional group that reacts with a reactive species. In this example, both a polyoxometalate and a metal oxide are being secured to the polymer, and a different linking agent is selected for the each. Both linking agents include a first isocyanate group for attachment to the reactive functional group on the polymer. However, after the linking agents have been attached to the polymer, the isocyanate/trialkoxysilane linking agent has unreacted silyl groups that covalently attaches a metal oxide nanoparticle while the diisocyanate linking agent has an unreacted isocyanate group that covalently attaches a polyoxometalate. Because of the differences in the exposed functional groups, the attachment of different reactive species can occur stepwise under unique conditions. As shown, the metal oxide is attached under a first set of conditions and then the polyoxometalate is attached under a second set of conditions.

The linking agents are at least bifunctional, including a first reactive functional group for covalent attachment to the reactive functional group on the polymer and a second reactive functional group for covalent attachment to the oxygen on the reactive species, i.e., polyoxometalate or metal oxide. For example, where the polymer includes a hydroxyl group, it would be appropriate for the linking agent to include a first reactive functional group selected from the group consisting of isocyanate, isothiocyanate, acyl halide, carboxylic anhydride, carboxylic acid, alkyl halide, oxirane, and oxetane.

If a polyoxometalate is being attached, then the second functional group is preferably selected from the group consisting of isocyanate, isothiocyanate, acyl halide, carboxylic anhydride carboxylic acid alkyl halide, oxirane, and oxetane. Accordingly, a preferred bifunctional linking agent for polyoxometalate attachment is a diisocyanate. Non-limiting examples of aromatic diisocyanates include 2,4-toluene diisocyanate, and 4,4′-methylenebis(phenyl isocyanate). Non-limiting examples of aliphatic diisocyanates include 4,4′-methylenebis(cyclohexyl isocyanate), and tetramethylxylene diisocyanate.

If a metal oxide is being attached, then the second reactive functional group is preferably a trialkoxysilyl group. Accordingly, a preferred bifunctional linking agent for metal oxide attachment is 3-(triethoxysilyl)propyl isocyanate.

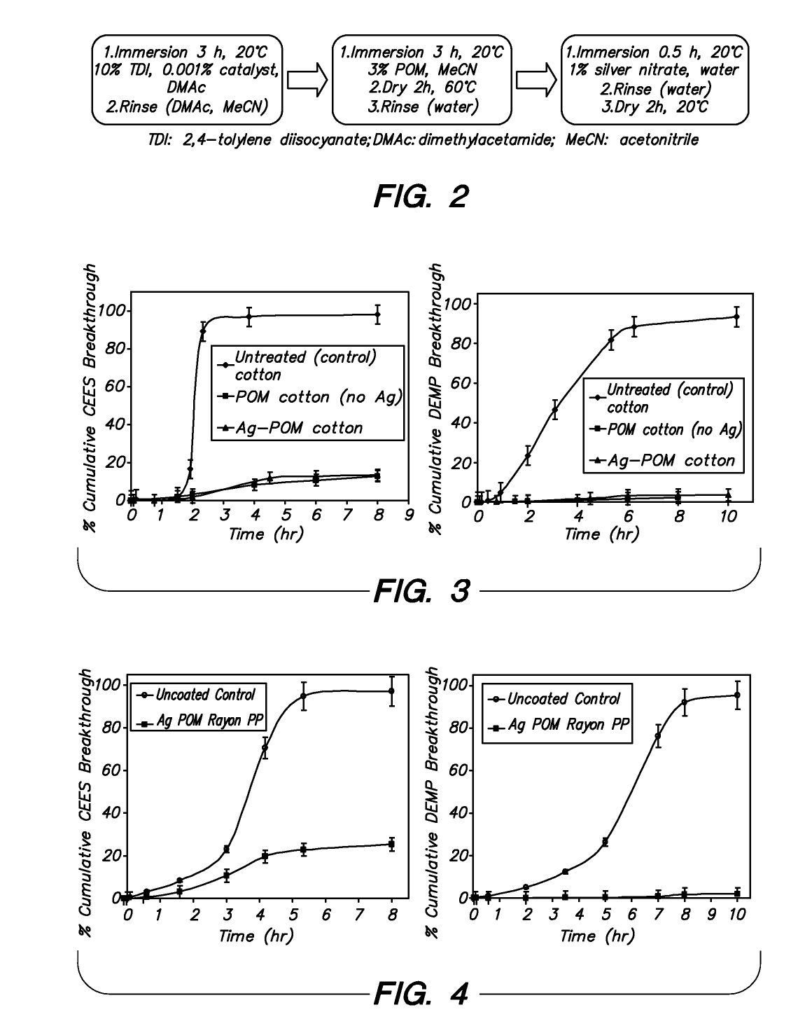

The process produces reactive fabrics with typical polyoxometalate contents ranging from 0.7 to 3.1 wt % and silver contents between 0.1 and 0.5 wt %, depending on the type of fabric, as determined by inductively-coupled plasma—atomic emission spectroscopy (ICP-AES). Since this process only employs simple immersion and drying steps, silver-polyoxometalate modified fabrics can be readily manufactured industrially by retrofitting existing infrastructure such as textile-dyeing equipment. FIG. 2 is a process flow diagram of the steps involved in grafting polyoxometalate onto a hydroxyl-containing polymer and depositing silver on the polyoxometalate.

The capabilities and methods of the invention may be further understood and illustrated by the following detailed examples. These examples describe specific embodiments of the invention, but should be taken to limit the scope of the invention.

### EXAMPLE 1

### Polyoxometalate Synthesis

A polyoxometalate complex, H₅PV₂Mo₁₀O₄₀, was synthesized as follows. V₂O₅ (1.20 mol) was suspended in 2.0 L water, and the mixture was heated to 60° C. Na₂CO₃ (1.20 mol) was slowly added, and the solution was heated to reflux for 1 hour. After adding 1 mL 30% H₂O₂ the solution was maintained at reflux for an additional 1 hour. The solution was filtered, and the filtrate was combined with MoO₃ (12.00 mol). The resulting mixture was heated to reflux, and additional Na₂CO₃ (1.80 mol) and concentrated H₃PO₄ (1.20 mol) were added sequentially. After 3 hours at reflux, the resulting solution was cooled to room temperature and diluted with water to a total volume of 40 mL, giving 0.3 M H₅PV₂Mo₁₀O₄₀.

A second polyoxometalate complex, the silver salt of polyoxovanadomolybdate Ag₅PV₂Mo₁₀O₄₀, was also synthesized as follows. Silver nitrate (48 mmol) was added to 40 mL of 0.3 M aqueous H₅PV₂Mo₁₀O₄₀ at room temperature. The resulting precipitate was filtered, washed with water and diethyl ether, and dried to afford 5.07 g of silver POM.

Both of the polyoxometalate materials were characterized using FTIR and UV/vis spectroscopy; all spectra were in good agreement with data reported in the literature. Swatches of 100% cotton, 100% rayon, 40/60% wood pulp/polyester blend, and 40/60% rayon/polypropylene blend were used to produce reactive fabrics.

### EXAMPLE 2

### Polyoxometalate Grafting with Tolylene Diisocyanate

Three 1½ in² fabric swatches were immersed in 50 mL of a 10 wt % solution of 2,4-tolylene-diisocyante in dry N,N-dimethylacetamide (DMAc) in a 250 mL round bottom flask. 50 μL of dibutyltin dilaurate were added, and the contents of the flask were stirred at ambient temperature for 4 hours. The swatches were then removed and thoroughly washed with DMAc, diethyl ether, and acetonitrile. The swatches were then stirred at ambient temperature for 8 hours with 50 mL of a 3 wt % solution of polyoxometalate (e.g. phosphomolybdic acid) in acetonitrile. The swatches were then removed, padded dry, and washed with water until no more polyoxometalate could be detected in the washes (typically 4 washes). The swatches were finally dried in ambient air.

### EXAMPLE 3

### Silverization of Polyoxometalate-Grafted Fabric

A silver treatment was carried out on the material prepared according to Example 2, as follows. The polyoxometalate-functionalized fabrics were added to a 30 mL aqueous silver nitrate solution (1 wt %), washed with deionized water and finally dried under vacuum.

### EXAMPLE 4

### Microbial Challenge of the Polyoxometalate-Grafted Fabric

A sample of the fabric of Example 3 was cut into quarters and placed into separate Petri dishes. A control (unmodified fabric) was cut to match the size of the sample pieces being tested. The bacteria of choice was diluted with phosphate buffered saline to obtain a 10⁶ CFU/mL inoculating solution. The samples and controls were inoculated with 100 μL of bacterial suspension. After a defined contact time, the sample was placed in 4.5 mL of D/E broth and sonicated for ten minutes. The cloth was aseptically removed from the test tube, and the liquid was serially diluted and plated out on its appropriate medium and incubated at 37° C. for 24 hours. The plates were then counted for colonies if any were present.

### EXAMPLE 5

### Chemical Challenge of the Polyoxometalate-Grafted Fabric

A modification of a standard military test (TOP 8-2-501) for agent breakthrough was utilized to test the resistance to penetration and breakthrough of chemical agents provided by the POM-modified fabrics. The chemical agents tested included 2-chloroethyl ethyl sulfide (CEES) and diethyl methylphosphonate (DEMP) as chemical warfare agent surrogates.

Circular swatches were placed inside a 2 cm diameter glass tube. The ends of the tube were capped and argon gas was flowed through the cloth. The Argon flowing through the cloth was bubbled through a 200 mL round bottom flask, containing 100 mL of dry toluene. A 20 μL drop of the chemical agent surrogate (CEES, DEMP) was placed directly onto the fabric surface in the center of the swatch. The concentration of the agent dissolved within the toluene was quantified using GC-MS at regular time intervals.

### EXAMPLE 6

### Cytotoxicity of the Polyoxometalate-Grafted Fabric

An agar overlay test was performed on modified fabric samples to evaluate the cytotoxicity of diffusible components from materials or solutions on cell culture monolayers. To verify that the modified textiles are safe to wear prior to and after exposure to toxic chemicals, modified cloth samples (pristine and challenged with CEES and DEMP, 20 μL each) were tested. An agar layer was added over cell monolayers (mouse embryo fibroblast, clone L-929) to act as a cushion to protect the cells from mechanical damage. The samples were then placed on top of the agar layer, and the cells were incubated. Cytotoxicity, if present, is scored as the degree of cellular damage or cytopathic effects. The following grades were given based on the description of the zone: Grade 0—no detectable zone around or under the sample, Grade 1—some malformed or degenerate cells under the sample, Grade 2—zone limited to area under the sample, Grade 3—zone extends 0.5 cm to 1.0 cm beyond the sample, and Grade 4—zone greater than 1 cm in extension from sample, but not involving the entire well. The sample met ISO/USP requirements (ISO 10993-5, USP 87) if none of the cell culture exposed to the sample showed greater than a mild reactivity (Grade 2). All the tested samples received a passing score of 2, proving that no toxic materials are released from the cloth, and also indicating that the chemical agents are in fact degraded.

### EXAMPLE 7

### Silver-Polyoxometalate Modified Fabrics Detoxify Hazardous Chemicals

A modification of a standard military test (TOP 8-2-501) was used to test the ability of the polyoxometalate-grafted fabric of Example 3 to detoxify hazardous chemicals. The concentration of the chemical agents dissolved within toluene was quantified using GC-MS at regular time intervals.

FIG. 3 shows the cumulative breakthrough behavior for CEES and DEMP on polyoxometalate modified cotton (no silver) compared to unmodified cotton as control. While significant agent penetration was observed with the controls after a two-hour challenge, the polyoxometalate modified samples very effectively prevented breakthrough of both surrogates. The treated samples provided protection for at least 8 hours, which demonstrates the efficiency of polyoxometalates.

In order to determine the influence of silver ions on the breakthrough behavior, silver-polyoxometalate modified cotton was challenged with CEES and DEMP. As seen in FIG. 3, the penetration protective properties are virtually unchanged when comparing polyoxometalate treated cotton with and without silver.

Several cellulose-based synthetic blends were similarly modified and tested. Silver-polyoxometalate modified wood pulp/polyester and rayon/polypropylene blends were tested for CEES and DEMP breakthrough in the same fashion as the cotton samples. When modified with silver and polyoxometalate, both blends provided breakthrough protection. The rayon/polypropylene blend showed properties similar to (for CEES) or even exceeding (for DEMP) the performance of modified cotton. FIG. 4 is a graph of the cumulative breakthrough behavior for CEES and DEMP on polyoxometalate modified fabric made from a rayon/polypropylene blend.

### EXAMPLE 8

### Silver-Polyoxometalate Modified Fabrics Catalytically Degrade Chemicals

In order to verify that the toxic chemicals are in fact degraded and not just strongly adsorbed, experiments were conducted to establish the occurrence of a chemical degradation reaction and prove that this reaction is catalytic in nature. To establish the presence of a chemical reaction, and to clearly distinguish from a mere physical adsorption, the polyoxometalate was monitored for a color change typically associated with oxidation reactions. A 20 μL drop of pure CEES was spotted onto a polyoxometalate-treated cotton swatch as well as onto an untreated cotton swatch as control. A reddish-brown spot appeared on the polyoxometalate-treated fabric after only 1-2 minutes, indicative of a reaction. No visible changes could be observed on the control.

Furthermore, to examine whether the immobilized polyoxometalate degraded toxic chemicals stoichiometrically (and would thus quickly saturate) or catalytically, the turnover numbers for the degradation reaction were determined. The turnover number is expressed as the amount of agent degraded (in mol) per amount of polyoxometalate (in mol). The amounts of degraded agent were determined from the breakthrough experiments, as the difference between polyoxometalate-modified and control cloth after 8 hours. The amount of polyoxometalate was determined by elemental analysis. As summarized in Table 1, turnover numbers exceeding 800 were observed, which clearly establishes a catalytic degradation mechanism.

TABLE 1
Catalytic turnover numbers for polyoxometalate-modified fabrics.

| | total POM | | | turnover |
|---|---|---|---|---|
| Fabric | (μmol) | Agent degraded (μmol) | | (mol_{agent}/mol_{POM}) |
| cotton | 1.731 | CEES | 145.190 | 83.89 |
| | | DEMP | 122.717 | 70.90 |
| rayon/PP blend | 0.151 | CEES | 123.321 | 818.24 |
| | | DEMP | 128.166 | 850.31 |
| wood pulp/PE | 0.249 | CEES | 71.238 | 286.61 |
| blend | | DEMP | 101.019 | 406.43 |

### EXAMPLE 9

### Modified Fabric Destroyed CWA Surrogates and a Common Pesticide in Liquid Phase

In order to determine the decontaminating properties of the fabric challenged in the liquid phase, cloth samples were exposed to small 200 μL drops of 1000 ppm solutions of agents chloroethyl phenyl sulfide (CEPS, H-agent surrogate), diphenyl chlorophosphonate (DPCP, G-agent surrogate), and Parathion, a commonly used pesticide in dichloromethane. After an incubation period of 30 min, the samples were extracted with dichloromethane, and the extracts were tested for recovered agent using GC. Untreated (no polyoxometalate) cotton pieces were used in a similar fashion as controls. Reductions of up to 95% were observed (see Table 2) on the modified textiles compared to the control samples.

TABLE 2
Decontamination of chemical agents
(1.5 in²; 200 μL of 1000 ppm agent)

| | Agent recovered after 30 min | | Reduction after |
|---|---|---|---|
| Chemical agent | POM | control | 30 min [%] |
| CEPS | 51 | 234 | 78 |
| DPCP | 87 | 388 | 78 |
| parathion | 32 | 597 | 96 |

### EXAMPLE 10

### Self-Detoxifying Properties Over Extended Periods

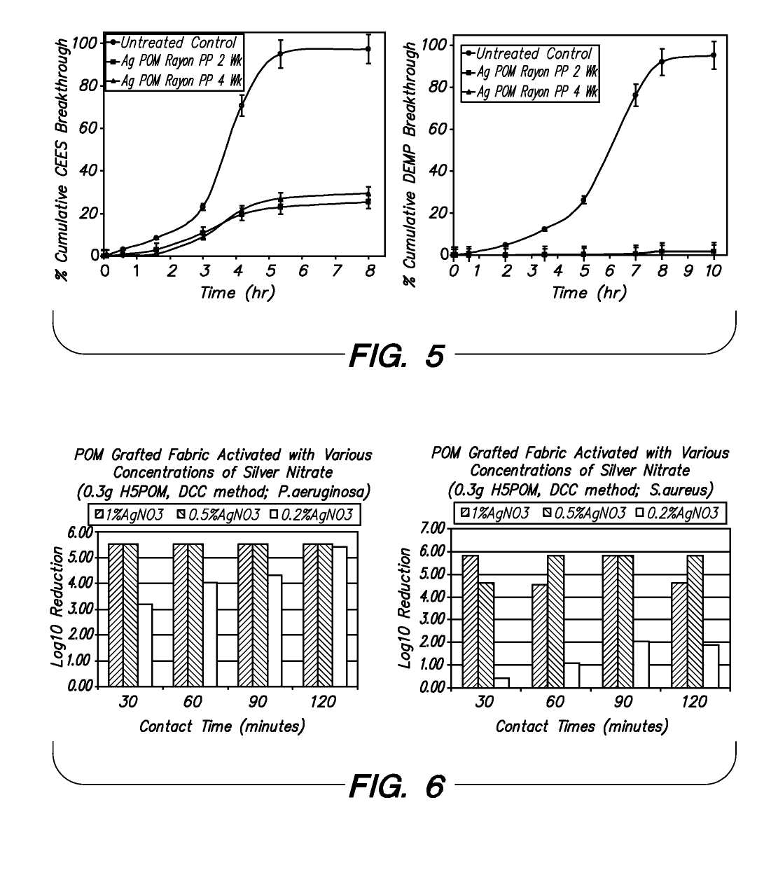

In order to establish long-term efficiency, and thus allow for prolonged storage of modified fabrics, we examined the chemical breakthrough behavior for CEES and DEMP on silver-polyoxometalate modified rayon/polypropylene blend that had been stored under ambient conditions for extended periods of time. As seen in the graph of FIG. 5, breakthrough protection is completely preserved after storage for several weeks. This result also indicates stabilization of the polyoxometalate through immobilization onto the textile fiber, as opposed to free polyoxometalate, which loses a high degree of activity after being exposed to ambient air.

### EXAMPLE 11

### Silver-Polyoxometalate Modified Textiles are Active Antimicrobials

To assess the antimicrobial properties of the reactive fabrics, Gram-positive Staphylococcus aureus and Gram-negative Pseudomonas aeruginosa were used as representative test strains. Samples of silver-polyoxometalate modified cotton and untreated cotton as a control were suspended in phosphate buffer and inoculated with microorganisms (typically 10⁶ CFU/mL), and placed on a shaker. Aliquots were taken every 30 minutes for two hours and plated on nutrient agar for enumeration by plate counting. To quantify bacterial reduction, the numbers for modified textiles were compared to the control.

The most active samples gave a 6 Log₁₀ CFU reduction corresponding to a 100% kill rate for both test strains after only 20 min contact time. In order to assess the effect of silver nitrate concentration on the overall antimicrobial activity, a series of experiments were carried out using 10⁶ CFU/mL of P. aeruginosa and S. aureus as inoculum. As expected, higher initial AgNO₃ concentrations (>0.5%) led to more active materials. The results are summarized in the bar graph of FIG. 6.

### EXAMPLE 12

### Mechanical Properties of Silver-Polyoxometalate Modified Fabric

In order to establish that the treatment did not have any adverse affects on the physical and mechanical properties of the fabric, mechanical testing was performed on samples of both modified and unmodified cotton. The first test, ASTM D4032, is a measurement of stiffness and was performed on an Instron 5569. The test involved a plunger forcing the samples through an orifice in a platform. The maximum force required was recorded and is an indication of the fabric stiffness. The results of the experiment showed that the treatment process only had a marginal effect on the fabric stiffness. The maximum load for the treated and control fabrics were determined to be 0.092 and 0.082 lbf, respectively, indicating a <10% increase in stiffness.

The second test, ASTM D5034, was performed to determine the tensile strength of modified and unmodified samples. A sample was clamped over an expandable diaphragm and the diaphragm was expanded by fluid pressure to the point of specimen rupture. The force required to break the fabric and average elongation at break were recorded. The results of this test again demonstrated that the modification had a minimal effect on the tensile strength. The breaking strength and maximum elongation were 29.06 lbs (61.08% elongation) for the modified samples, and 25.96 lbs (64.44% elongation) for the control samples. Both these results indicate that fabric properties are not altered to any considerable extent.

### EXAMPLE 13

### Metal Oxide Nanoparticle Grafting on Cotton Cloth

A simple two-step protocol was used to covalently attach metal oxide nanoparticles onto the surface of loosely woven cotton cloth. Three square swatches of the cotton cloth material, measuring 2.5 cm on each side were cut, and refluxed with 3 mL 3-(triethoxysilyl)propyl isocyanate, and 150 μL dibutyltin dilaurate in 50 mL dimethyl acetamide. The cloth was then thoroughly washed with diethyl ether and hexanes, and completely dried under reduced pressure at room temperature overnight. The three dry silyl modified cotton swatches were refluxed with 600 mg of calcium oxide nanoparticles in 50 mL toluene for 2 hr. The fabric was thoroughly washed with toluene, diethyl ether and hexanes and dried in a vacuum oven overnight at 84° C., to obtain cotton swatches with 12 wt. % nanoparticles covalently grafted. The weight percent grafted onto the fabric was determined through difference in the weights of the cloth swatch before and after nanoparticle attachment and confirmed via ICP-OES analysis.

### EXAMPLE 14

### Metal Oxide Nanoparticle Grafting on Polypropylene Material

ProVent™ 1000 (Kappler, Inc.; Guntersville, Ala.) chemical and biological barrier material is comprised of an inert polypropylene polymer. Activation of the inert ProVent 1000 surface was conducted by placing a 2.5 cm×2.5 cm swatch of ProVent 1000 into a Plasma Cleaner/Sterilizer, which utilizes a stream of highly charged and energetic particles to generate reactive oxygen species (ROS) on the surface of the ProVent 1000 swatch. This activated swatch was immediately utilized to covalently graft calcium oxide nanoparticles using an identical procedure as that for the cotton cloth in Example 13. The ProVent 1000 swatches were determined to have 3 wt. % of the covalently attached nanoparticles (determined through difference in weight before and after nanoparticle attachment).

### EXAMPLE 15

### Chemical Agent Challenge of Metal Oxide Grafted Cotton

A modification of a standard military test (TOP 8-2-501) for agent breakthrough was utilized to test the resistance to penetration and breakthrough provided by nanoparticle attached cotton cloth swatches, by placing a square swatch of the nanoparticle modified cotton inside a 2 cm diameter glass tube, capping the ends and flowing argon gas through the cloth. The Argon flowing through the cloth bubbled through a 200 mL round bottom flask, containing 100 mL of dry Toluene. A 20 μL drop of the chemical agent surrogate was placed directly onto the cotton swatch surface in the center of the swatch. The concentration of the agent dissolved within the toluene was quantified using GC-MS at regular time intervals.

The nanoparticle modified cotton cloth significantly prevented breakthrough of 2-chloroethyl ethyl sulfide (CEES), an H agent surrogate, whereas regular cotton allowed CEES to completely break through, as shown in FIG. 8. It also appears that the covalent attachment protects the nanoparticles from atmospheric moisture based deactivation that occurs rapidly and almost completely when unmodified metal oxides are exposed to the atmosphere.

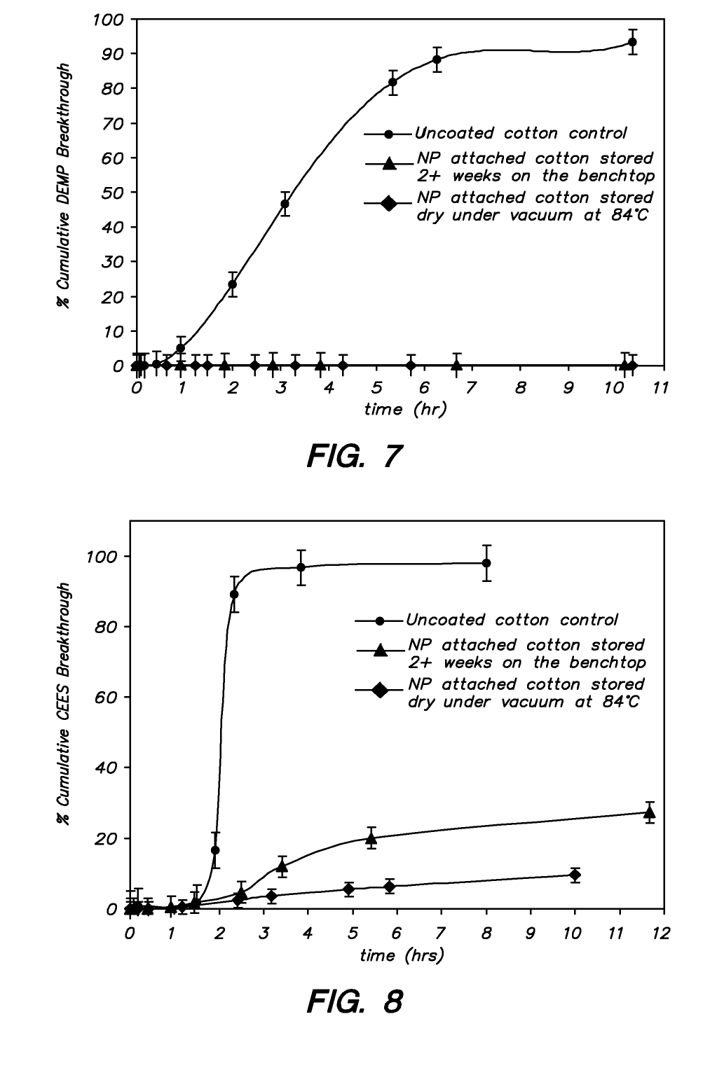

FIG. 7 is a graph showing the breakthrough behavior of diethyl methyl phosphonate (DEMP), a G agent surrogate, using the nanoparticle attached cotton swatches. As expected, the DEMP breaks through the unmodified cotton swatch almost completely in a reasonably short time, while the nanoparticle attached cloth almost completely prevents the breakthrough of DEMP. The breakthrough behavior is nearly identical for both the freshly prepared (vacuum and heat treated) nanoparticle cotton or nanoparticle cotton which has been in prolonged contact with atmospheric moisture. This indicates that the nanoparticles may be protected against atmospheric moisture degradation when covalently embedded within the cotton cloth.

### EXAMPLE 16

### Chemical Agent Challenge of Metal Oxide Grafted Polypropylene Material

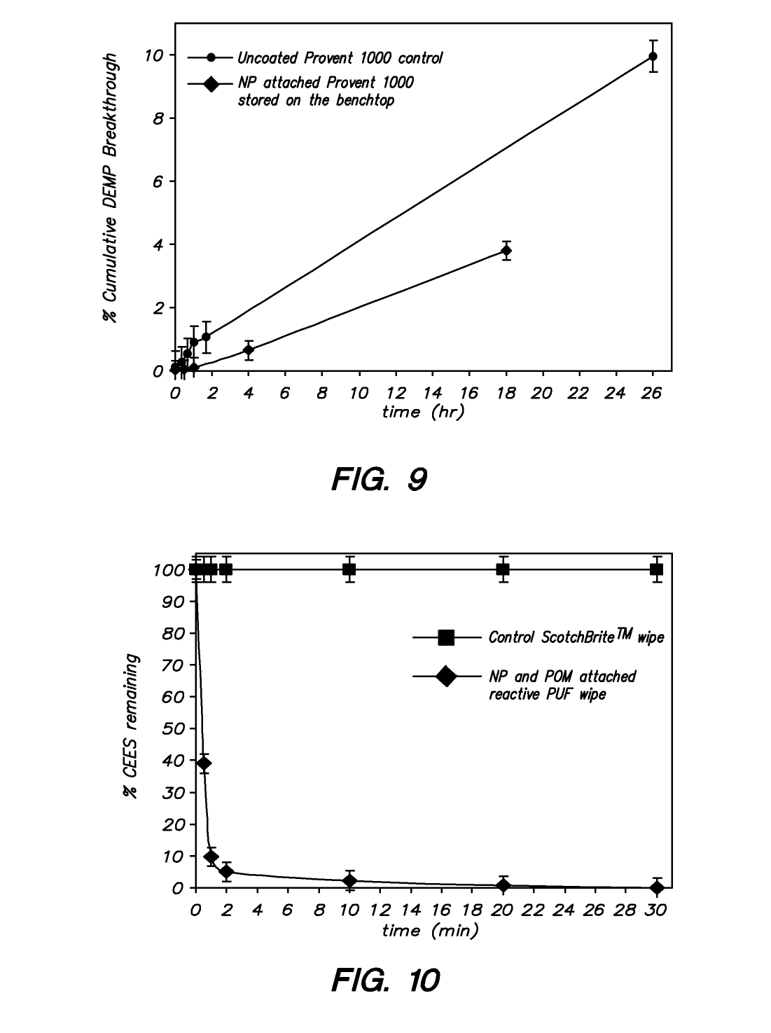

The nanoparticle attached ProVent 1000 cloth was treated with G agent surrogate (DEMP) and the cumulative breakthrough experiment was conducted as done for the cotton swatches in Example 15. The ProVent 1000 polypropylene material appears to significantly retard the penetration of the agent even when there are no nanoparticles attached to its surface. This is expected because ProVent 1000 is a barrier material and is commercially available as the basic material for chemical and biological protective garments. However, the nanoparticle attached ProVent 1000 clearly provides even better agent penetration and breakthrough resistance as shown in FIG. 9. A 50% reduction in the agent penetration through ProVent 1000 can therefore be achieved by covalently grafting merely 3 wt. % nanoparticles onto its surface.

### EXAMPLE 17

### Metal Oxide Modified Fabrics Chemically Decompose Chemicals

In order to show that the nanoparticle attached cloth swatches actively decompose the chemical agent surrogates, and not merely adsorb or retard their passage through the cloth, the nanoparticle attached cotton cloth swatch was treated with a 5 μL droplet of paraoxon, which is a toxic industrial chemical and pesticide. The cotton swatches utilized had been stored on the bench for more than 2 weeks in direct contact with atmospheric moisture. The area on the nanoparticle cloth to which the paraoxon is applied turned into a yellow spot, due to the hydrolysis of the paraoxon to p-nitrophenolate anion, which is intensely yellow in color. Conversely, a control swatch of regular cotton treated with 5 μL paraoxon did not show the development of a yellow spot, indicating that no hydrolytic decomposition of the paraoxon occurred. This experiment, in combination with the breakthrough results described above, strongly suggest that the nanoparticles which are covalently attached to the cloth actively hydrolyze chemical agents and do not merely adsorb them.

### EXAMPLE 18

### Preparing Polyurethane Foam with Covalently Attached Polyoxometalates and Metal Oxide Nanoparticles

A 2 cm³ cube of commercially available off the shelf kitchen surface cleaning soft polyurethane foam sponge (ScotchBrite™, 3M, St. Paul, Minn.) was placed in a Plasma Cleaner/Sterilizer and subjected to a stream of highly energetic charged particles for 3 minutes to generate reactive oxygen species (ROS) throughout the surface (interior pores and exterior). The sponge was immediately placed in a beaker containing 1 mL of 3-(triethoxysilyl)propyl isocyanate and 1 mL of 2,4-toluene diisocyanate dissolved in 100 mL of dry toluene under an argon atmosphere for 3 hours at room temperature. The resultant silyl and isocyanate group containing sponge was thoroughly washed with organic solvents, and dried under vacuum at room temperature for 1 hour. The resultant dry cube was refluxed with a dry toluene suspension of 600 mg of calcium oxide nanoparticles under argon for 3 hours. The resultant nanoparticle-modified polyurethane foam was washed thoroughly with organic solvents and dried under vacuum at room temperature for 1 hour. The cube was then placed in a beaker containing 2 grams of phosphomolybdic acid dissolved in 100 mL of dry THF under argon for 24 hours. This resultant metal oxide nanoparticle and polyoxometalate grafted polyurethane foam sponge cube was thoroughly washed with dry THF and dry ether and dried at room temperature under vacuum overnight. The resultant yellow sponge had 4 wt. % polyoxometalate and 5 wt. % metal oxide nanoparticles attached covalently to its surface, determined through weight difference.

### EXAMPLE 19

### Polyoxometalate and Metal Oxide-Modified Polyurethane Foam Chemically Decomposes Chemicals

The reactivity of the sponge was confirmed by addition of a 6 μL drop of 2-chloroethyl ethyl sulfide (CEES) an H agent simulant to the surface of the wipes. CEES is a colorless liquid, and can be oxidized by the polyoxometalate groups while the polyoxometalate itself gets reduced. The oxidized CEES products are colorless, while the reduced polyoxometalate is intensely red-brown in color. An intense red-brown spot immediately developed on the metal oxide nanoparticle and polyoxometalate sponge, while there was no color change on the starting ScotchBrite™ sponge. The production of this deep red-brown spot upon addition of CEES confirms that the sponge absorbs the agent surrogate and reactively decomposes it.

Stainless steel #616 coupons were used as an example of a non-porous relevant surface. A 2 μL drop of the neat chemical agent surrogate was placed onto the coupon, and wiped off gently with a slight dabbing action with a polyurethane metal oxide nanoparticle and polyoxometalate foam wipe measuring 2 cm×2 cm×3 mm. The wipe was allowed to react with the absorbed agent for various time intervals (ranging from 30 seconds to 30 minutes), then immersed in 10 mL of HPLC grade toluene, and vigorously agitated on a vortex shaker for 20 seconds. The wipe was then discarded, and the toluene into which the remaining agent was extracted was analyzed using GC-MS. In addition, the decontaminated coupon surface was examined under a microscope to check for residues or damage. Lastly, the decontaminated coupon was extracted with 10 mL of toluene and the extract analyzed using GC-MS.

The metal oxide nanoparticle and polyoxometalate group attached reactive wipe was first utilized for decontamination of the H agent surrogate 2-chloroethylethyl sulfide (CEES) using the above procedure. The wipe performs its function admirably in that it can almost completely destroy CEES within a few minutes (less than 20 minutes) of uptake from the stainless steel coupon surface. In contrast, the control ScotchBrite™ wipe without metal oxide nanoparticle and polyoxometalate does not destroy CEES at all based on the nearly 100% recovery even after 30 minutes, as shown in FIG. 10.

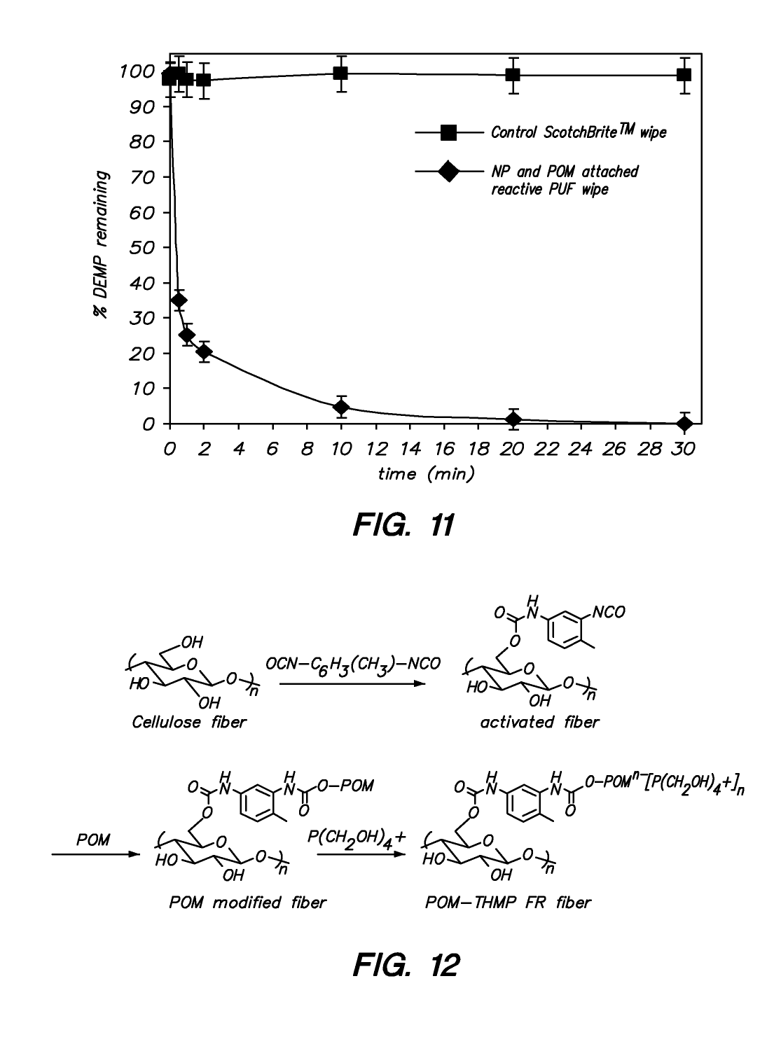

The metal oxide nanoparticle and polyoxometalate group attached reactive wipe was also utilized for decontamination of the G agent surrogate dimethyl ethyl phosphonate (DEMP) using the above procedure. The wipe almost completely destroyed DEMP within a few minutes (less than 20 minutes) of uptake from the stainless steel coupon surface, as shown in FIG. 11. In contrast, the control ScotchBrite™ wipe does not destroy DEMP based on the nearly 100% recovery even after 30 minutes.

Under visual inspection with an optical stereo microscope, the decontaminated stainless steel coupon did not have any resides, either solid fibers or particles, or liquid films on the treated area, nor any scratches or evidence of abrasion around the treated area. This is excellent evidence to suggest that the foam wipes have superior absorbency and can take up the entire deposited chemical agent into its interior rapidly. No traces of residual chemical agent (either DEMP or CEES) was observed (GC-MS) via toluene extraction of treated surfaces.

### EXAMPLE 20

### Fabric Modified by Covalent Attachment of Titanium Dioxide Nanoparticles and Incorporation of Dye

Reactive nanoparticle-modified fabrics were prepared using a bifunctional isocyanate-alkoxy silane linker to covalently attach titanium dioxide nanoparticles to the fibers of a cotton fabric. Swatches of the fabrics were immersed in a solution of triethoxysilylpropyl isocyanate (10 wt %) and dibutyltin dilaurate (0.2 wt %) in dimethylacetamide for 6 hours at ambient temperature. The fabrics were then rinsed with acetonitrile, and subsequently immersed in a 2 wt % dispersion of titanium dioxide nanoparticles in acetonitrile for 6 hours at ambient temperature. The swatches were then dabbed dry and heated to 80° C. under a vacuum over night. The swatches were rinsed multiple times with acetonitrile to remove any unbound nanoparticles, and dried in ambient air. The dried TiO₂ modified swatches were then immersed in an ethanol/water solution of methyl red (0.1-1.0 mg/mL) neutralized with NaOH (0.1N NaOH was titrated into the dye solution until the persistent color changed from red to yellow). The swatches were dried at 80° C. under a vacuum over night.

### EXAMPLE 21

### Visual Detection of Hydrolytic Degradation of Contaminants

The Reactive nanoparticle/dye-modified fabric of Example 20 was exposed to CEES (an H-agent surrogate), DEMP (a G-agent surrogate), and malathion (a V-agent surrogate). A 5 μL doplet of each agent was placed on separate swatches of the modified fabric, as well as on control swatches that were modified only with the dye. Each of the swatches was initially yellow. The challenge with CEES produces an orange to red coloration. The challenge with DEMP produced a clearly visible color change from yellow to red within less than 5 minutes. The challenge with malathion produced a clearly visible color change from yellow to red within less than one minute. No color change was seen in any of the control swatches, nor a separate swatch of the nanoparticle/dye-modified fabric that did not receive a challenge. These results indicate that the TiO₂ nanoparticles hydrolytically degraded each of the agents with concurrent acid liberation, which caused a color change in the dye.

### EXAMPLE 22

### Synthesis of POM-THMP Modified Textile

Swatches of cotton cloth material (1.5″×1.5″) were immersed in a solution of 2,4-toluene diisocyanate (5 mL), and dibutyltin dilaurate (150 μL) in dimethyl acetamide (50 mL) for 8 h. The swatches were thoroughly washed with diethyl ether and hexanes, and completely dried under reduced pressure at room temperature overnight. The diisocyanate functionalized swatches were then immersed in a solution of phosphomolybdic acid in acetonitrile (50 mL saturated solution) at ambient temperature. After stirring overnight, the swatches were dried at 50° C., washed with deionized water, and finally dried in ambient air. As determined by elemental analysis (ICP-AES), this process produced a POM content between 3 to 5 wt % immobilized on the fabric.

The POM-modified swatches were immersed in a solution of tetrakis(hydroxymethyl)-phosphonium chloride (6 mL of an 80% solution) in water (50 mL) for 10 min at ambient temperature with gentle agitation. THMP was incorporated onto the POM through ion exchange. The fabrics were padded dry and cured at 80-110° C. for 20 min. The resulting fabrics are stable under ambient condition for extended periods of time without losing their activity. Furthermore, immersion in both tap water (to simulate laundering) and toluene (to simulate dry cleaning) does not result in any leaching of POM and THMP; the modified fabrics fully retain their activity.

FIG. 12 is a schematic diagram of the reaction of hydroxyl groups naturally present on cellulose fibers with a 2,4-toluene diisocyanate cross linker, subsequent attachment of polyoxometalate on the activated fiber, and the ion exchange of THMP onto the POM.

The methods and materials of the present invention have shown that chemical and biological agent protection can be achieved through reactive decontamination. Metal oxides and polyoxometalates are stabilized against moisture based deactivation. Fabrics modified in accordance with the invention experience no significant change in physical characteristics and can be washed and stored without loss of activity. These materials are non-cytotoxic before and after exposure to chemical agents. Some or all of these properties make the invention suitable for use in surgical environments, agriculture, military, industrial, and household disinfection.

The terms “comprising,” “including,” and “having,” as used in the claims and specification herein, shall be considered as indicating an open group that may include other elements not specified. The term “consisting essentially of,” as used in the claims and specification herein, shall be considered as indicating a partially open group that may include other elements not specified, so long as those other elements do not materially alter the basic and novel characteristics of the claimed invention. The terms “a,” “an,” and the singular forms of words shall be taken to include the plural form of the same words, such that the terms mean that one or more of something is provided. The terms “at least one” and “one or more” are used interchangeably. The term “one” or “single” shall be used to indicate that one and only one of something is intended. Similarly, other specific integer values, such as “two,” are used when a specific number of things is intended. The terms “preferably,” “preferred,” “prefer,” “optionally,” “may,” and similar terms are used to indicate that an item, condition or step being referred to is an optional (not required) feature of the invention.

What is claimed is:

1. A method of modifying a polymer, comprising:
reacting a first reactive functional group of at least one bifunctional linking agent with a reactive moiety of the polymer to form a covalent bond therebetween; and
reacting at least one oxygen atom of at least one reactive species with a second reactive functional group of the at least one bifunctional linking agent to form a covalent bond therebetween.

2. The method of claim 1, wherein the first reactive functional group is selected from the group consisting of isocyanate, isothiocyanate, acyl halide, carboxylic anhydride, carboxylic acid, ester, alkyl halide, oxirane, and oxetane.

3. The method of claim 1, wherein the second functional group is selected from the group consisting of isocyanate, isothiocyanate, acyl halide, carboxylic anhydride carboxylic acid, ester, alkyl halide, oxirane, and oxetane.

4. The method of claim 1, wherein the at least one reactive species is selected from the group consisting of a heteropolyacid, a polyoxometalate, or a combination thereof.

5. The method of claim 1, wherein the first and second reactive functional groups are independently selected from the group consisting of isocyanate, isothiocyanate, acyl halide, carboxylic anhydride, carboxylic acid, ester, alkyl halide, oxirane, and oxetane;
and wherein the at least one reactive species is selected from the group consisting of a heteropolyacid, a polyoxometalate, or a combination thereof.

6. The method of claim 5, wherein the at least one reactive species is a polyoxometalate.

7. The method of claim 5, wherein the at least one reactive species is a polyoxometalate selected from the group consisting of Keggin-type polyoxometalates defined by the general formula (R^{m+})_{y}[Xⁿ⁺M₁₂O₄₀]⁽⁸⁻ⁿ⁾⁻ where m and n are an integer, y is defined as y=(8−n)/m, X is selected from phosphorus and silicon, M is selected from molybdenum, tungsten, vanadium, and combinations thereof, O is oxygen, and R is selected from the group consisting of hydrogen, silver, ammonium, quaternary ammonium, and combinations thereof.

8. The method of claim 1, wherein the at least one reactive species is a polyoxometalate selected from the group consisting of H₅PV₂Mo₁₀O₄₀, Ag₅PV₂Mo₁₀O₄₀, and H₃PMo₁₂O₄₀ and combinations thereof.

9. The method of claim 1, wherein the bifunctional linking agent is a diisocyanate.

10. The method of claim 1, wherein the bifunctional linking agent is an aromatic diisocyanate selected from the group consisting of 2,4-toluene diisocyanate, 2,4-toluene diisocyanate, 4,4′-methylenebis(phenyl isocyanate), tetramethylxylene diisocyanate.

11. The method of claim 1, wherein the bifunctional linking agent is toluene diisocyanate.

12. The method of claim 1, wherein the at least one reactive species is a metal oxide.

13. The method of claim 12, wherein the second reactive functional group is a trialkoxysilyl group.

14. The method of claim 13, wherein the first reactive functional group is selected from the group consisting of isocyanate, isothiocyanate, acyl halide, carboxylic anhydride, carboxylic acid, alkyl halide, oxirane, and oxetane.

15. The method of claim 12, wherein the metal oxide is selected from the group consisting of TiO₂, MgO, ZnO, CaO, Al₂O₃, and combinations thereof.

16. The method of claim 12, wherein a majority of the metal oxide nanoparticles have a particle size between 1 and 100 nanometers.

17. The method of claim 12, wherein the linking agent is (triethoxysilyl)propyl isocyanate.

18. The method of claim 1, further comprising:
hydroxylating the polymer through exposure to an oxygen plasma or ozone.

19. The method of claim 1, wherein the polymer is a polysaccharide selected from the group consisting of cellulose, starch, chitosan, chitin, and combinations thereof.

20. The method of claim 1, wherein the at least one reactive species includes both a polyoxometalate and a metal oxide, and wherein the at least one linking agent includes both a diisocyanate and an isocyanate/alkoxysilane.

21. The method of claim 20, wherein the diisocyanate and the isocyanate/alkoxysilane are simultaneously reacted with reactive moiety of the polymer to form an activated polymer.

22. The method of claim 21, further comprising:
reacting the metal oxide with alkosysilane groups of the activated polymer before reacting the polyoxometalate with isocyanate groups of the activated polymer.

23. The method of claim 1, further comprising:
immobilizing silver ions on the reactive species.

24. The method of claim 1, further comprising:
incorporating an indicator dye onto the polymer, wherein the dye exhibits a change in color in response to a change in pH caused by reacting a contaminant species at the reactive species.

25. A textile that has been modified in accordance with the method of claim 1.

26. A polymeric material, comprising:
at least one reactive species covalently linked to a polymer, wherein the reactive species is selected from the group consisting of heteropolyacid, a polyoxometalate, a metal oxide, and combinations thereof.

27. The material of claim 26, wherein the at least one reactive species includes a polyoxometalate.

28. The material of claim 26, wherein the at least one reactive species includes a metal oxide linked through a trialkoxysilyl group.

29. The material of claim 28, wherein the metal oxide is selected from the group consisting of TiO₂, MgO, ZnO, CaO, Al₂O₃, and combinations thereof.

30. The material of claim 26, wherein the at least one reactive species includes both a polyoxometalate and a metal oxide, and wherein the at least one linking agent includes both a diisocyanate and an isocyanate/alkoxysilane.

31. The material of claim 26, further comprising:
silver ions immobilized on the reactive species.

32. The material of claim 26, further comprising:
a dye incorporated onto the polymer, wherein the dye exhibits a change in color in response to a change in pH caused by reacting a contaminant species at the reactive species.

33. The material of claim 27, further comprising:
a flame retardant attached to the polyoxometalate.
